(19) United States
(12) Patent Application Publication
(10) Pub. No.: US 2008/0019939 A1
(43) Pub. Date: Jan. 24, 2008

(54) Conditioner formulation

(75) Inventor: Gilles Verboom, St. Charles, IL (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: Alberto-Culver Company, Melrose Park, IL (US)

(21) Appl. No.: 11/490,403

(22) Filed: Jul. 20, 2006

(51) Int. Cl. A61K 8/89

(52) U.S. Cl. 424/70.122

(57) ABSTRACT

Provided is a conditioning formulation, which preferably includes one or more bis-alkoxy-terminated glycol-functionalized aminosilicones, one or more non-functionalized aminosilicones, one or more fatty alcohols, one or more alkyl quaternary ammonium salts, and water. Also provided is a method of conditioning keratinous fibers (e.g., hair), which includes applying to the keratinous fibers an effective amount of the formulation of the present invention and, optionally, removing (e.g., rinsing) at least a portion of the formulation therefrom.

### BACKGROUND OF THE INVENTION

Hair, e.g., human hair, becomes soiled primarily due to its contact with the surrounding environment and from the sebum secreted by the scalp. The soiling of hair causes it to have a dirty feel and an unattractive appearance. Consequently, people tend to use shampoo on the hair with frequent regularity, e.g., to remove excess soil and sebum. However, shampooing can leave the hair in a wet, tangled, and generally unmanageable state, and often leaves hair in a dry, rough, lusterless, or frizzy condition due to removal of the hair's natural oils and other natural conditioning and moisturizing components. Other sources of hair damage include subjecting the hair to, e.g., drying, heating, combing, styling, perming, coloring, and exposure to the elements.

Hair conditioners are often employed to restore damage to the hair caused by shampooing and other causes. Many different approaches have been developed to condition the hair. A common method of conditioning hair is by applying conditioning compositions, which include agents such as cationic surfactants and polymers, fatty compounds, oils, and silicone compounds. However, conventional conditioners can suffer from a number of drawbacks. For instance, it is difficult to formulate a conditioner, particularly a conditioner with a high fatty alcohol content, which remains stable under freeze-thaw conditions and/or elevated storage temperatures, and yet exhibits excellent conditioning properties such as, e.g., hair shine, softness, dry hair smoothness, hair strand alignment, and ease of combing.

A need exists for conditioning formulations, which are stable under freeze-thaw conditions and/or under elevated storage conditions, and yet exhibit excellent conditioning properties, e.g., hair shine, softness, dry hair smoothness, hair strand alignment (e.g., to minimize frizziness), and ease of combing. There is also a need for methods of using such formulations. The present invention provides such formulations and methods.

### BRIEF SUMMARY OF THE INVENTION

The present invention provides a conditioning formulation, which can be used as a rinse-off conditioner for treating keratinous fibers. In one embodiment, the present invention provides a composition, which includes one or more bis-alkoxy-terminated glycol-functionalized aminosilicones, one or more non-functionalized aminosilicones, one or more fatty alcohols, one or more alkyl quaternary ammonium salts, and water. The compositions of the present invention exhibit stable emulsion properties under freeze-thaw conditions and/or under conditions of storage at elevated temperature. In addition, the compositions of the present invention, when applied to the hair (e.g., as a rinse-off conditioner) impart hair with a silky wet feel.

The present invention also provides a method of conditioning keratinous fibers, which preferably includes applying to the keratinous fibers a conditioning-effective amount of the formulation of the present invention. The keratinous fibers can include mammalian hair, e.g., human hair.

### DETAILED DESCRIPTION OF THE INVENTION

The present invention provides a conditioning formulation, which can be used as a rinse-off conditioner, e.g., for treating keratinous fibers, and which is stable under freeze-thaw conditions and/or under conditions of storage at elevated temprature. In one embodiment, the present invention provides a composition, which includes one or more bis-alkoxy-terminated glycol-functionalized aminosilicones, one or more non-functionalized aminosilicones, one or more fatty alcohols, one or more alkyl quaternary ammonium salts, and water.

Any suitable bis-alkoxy-terminated glycol-functionalized aminosilicone, which includes combinations of one or more bis-alkoxy-terminated glycol-functionalized aminosilicones, can be used in the composition of the present invention. Suitable bis-alkoxy-terminated glycol-functionalized aminosilicones can include, e.g., one or more bis-C₁₃-C₁₅-alkoxy-terminated glycol-functionalized aminosilicones. For instance, the bis-alkoxy-terminated glycol-functionalized aminosilicones can include one or more bis-C₁₃-C₁₅-alkoxy-terminated propylene glycol-functionalized aminosilicones. Exemplary bis-alkoxy-terminated glycol-functionalized aminosilicones include one or more copolymers of the formula (I):

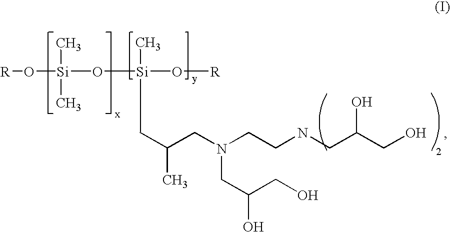

wherein each R is independently a C₁₃-C₁₅ alkyl, x is from about 25 to about 1000, and y is from 1 to about 200, and x and y preferably are chosen such that the nitrogen content exceeds 0.5%. Suitable bis-alkoxy-terminated glycol-functionalized aminosilicones also are described, e.g., in WO 03/066007. A particularly preferred bis-alkoxy-terminated glycol-functionalized aminosilicone of formula (I) is commercially is a product that is sold by Dow Coming under the trademark DC-8500™.

Any suitable non-functionalized aminosilicone, which includes combinations of one or more non-functionalized aminosilicones, can be used in the conditioner formulation of the present invention. For example, suitable non-functionalized aminosilicones can include one or more compounds of the formula (II):

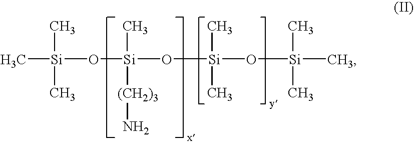

wherein the x′:y′ratio is about 1:50, and the molecular weight of the compound is at least about 16,000. Compounds of the formula (II), as defined above, are a particularly preferred class of non-functionalized aminosilicones for use in the conditioner formulation of the present invention. An exemplary non-functionalized aminosilicone of formula II is sold by Ciba Specialty Chemicals under the trademark Tinocare® Si Al. See also U.S. 2003/0157049.

Suitable non-functionalized aminosilicones also can include one or more compounds of the formula (III):

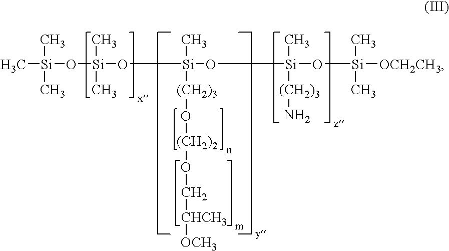

wherein x″ is from about 20 to about 100, y″ from about 2 to about 10, z″ is from 1 to about 5, m is from about 3 to about 5, and n is from about 5 to about 9. An exemplary non-functionalized aminosilicone of formula (III) is a product sold by Degussa under the trademark ABIL® Soft AF 100.

Suitable non-functionalized aminosilicones also can include one or more polyoxyalkylenated aminosilicone polymers of the (AB)ₙ type, wherein A is a polysiloxane block and B is a polyoxyalkylenated block, comprising repeating units of the formulae: [SiMe₂-O—(SiMe₂O)_{x′″}SiMe₂-R′—N(H)—R″—O(C₂H₄O)ₐ—(C₃H₆O)_{b}—R″—N(H)—R′—], wherein: (i) a is from about 1 to about 200, (ii) b is from 0 to about 200, (iii) each R′, which may be identical or different, is a divalent organic group which is linked to the adjacent silicon atom via a carbon-silicon bond and to the nitrogen atom, (iv) each R″, which may be identical or different, is a divalent organic group which is linked to the adjacent oxygen atom via a carbon-oxygen bond and to the nitrogen atom, and (v) x′″ is chosen such that the siloxane block represents between 50 and 95 mol % relative to the total weight of the polyoxyalkylenated aminosilicone polymer. Non-functionalized polyoxyalkylenated aminosilicone polymer of the (AB)ₙ type are described in U.S. Pat. No. 6,589,519. An exemplary non-functionalized polyoxyalkylenated aminosilicone polymer of the (AB)ₙ type is sold by GE Silicones under the trade name Silsoft A-843.

Any suitable fatty alcohol, which includes combinations of one or more fatty alcohols, can be used in the formulation of the present invention. Fatty alcohols used in formulation of the present invention preferably have from about 12 to about 20 carbon atoms, and more preferably include one or more C₁₆-C₁₈ alcohols. Non-limiting examples of fatty alcohols include, e.g., cetyl alcohol, stearyl alcohol, and combinations thereof. The formulation of the present invention preferably includes from about 3.0 wt % to about 8.0 wt % of one or more fatty alcohols (relative to the total weight of the formulation), and more preferably from about 4.0 wt % to about 7.0 wt % of one or more fatty alcohols, and most preferably from about 5.0 wt % to about 6.0 wt % of one or more fatty alcohols.

Any suitable quaternary ammonium salt, which includes combinations of one or more alkyl quaternary ammonium salts, can be used in the formulation of the present invention. Suitable alkyl quaternary ammonium salts can include, for example, compounds of the general formula (IV):

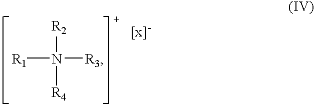

wherein R₁ and R₂ are the same or different and each is a linear or branched aliphatic substituent containing from 1 to about 30 carbon atoms, and R₃ and R₄ are the same or different and each is a linear or branched alkyl substituent containing from 1 to about 3 carbon atoms or an aralkyl substituent containing at least 7 carbon atoms, and X is an anion (e.g., halide (e.g., chloride, bromide), sulfate, sulfonate, phosphate, phosphonate, and the like). See also, e.g., US 2003/0157049. Preferred alkyl quaternary ammonium salts of general formula (IV) include one or more compounds of formula (IV), wherein at least one of R₁ and R₂ is an alkyl chain having at least about 16 carbon atoms. Exemplary alkyl quaternary ammonium salts, which can be used in the formulation of the present invention, include stearyl dimethyl benzyl ammonium chloride, cetrimonium chloride (e.g., Varisoft 300, sold by Degussa), and combinations thereof. The formulation of the present invention preferably includes one or more alkyl quaternary ammonium salts in an amount of from about 1.5 wt % to about 2.5 wt % (based on the total weight of the formulation).

Water preferably serves as an aqueous carrier or vehicle in the conditioning formulation of the present invention. Any suitable quantity of water can be used in the formulation of the present invention, e.g., from about 60 wt % to about 95 wt % water (e.g., from about 85% to about 95% water). Preferably, the formulation of the present invention includes from about 60 wt % water to about 90 wt % water, and more preferably from about 70 wt % water to about 90 wt % water, and most preferably from about 80 wt % water to about 90 wt % water. Preferably, deionized water is used in the formulation of the present invention.

The formulation of the present invention can further include one or more silicones. Suitable silicones can include, for example, volatile silicones (e.g., one or more cyclomethicones, one or more volatile linear silicones, and combinations thereof) and non-volatile silicones (e.g., non-volatile linear silicon, e.g., non-volatile dimethicones), and combinations thereof. Exemplary volatile cyclic silicones include cyclomethicones having from 3 to 7 silicon atoms (e.g.,as dimethylsiloxane units), e.g., from 4 to 5 silicon atoms (e.g., as dimethylsiloxane units). Exemplary volatile linear silicones include linear silicones having from 2 to about 9 silicon atoms (e.g., as dimethylsiloxane units). Preferably, the silicone used in the formulation of the present invention includes a combination of at least one non-volatile silicone and at least one volatile silicone. Exemplary silicones, which can be used in the formulation of the present invention, include one or more dimethicones, one or more hydroxyl-terminated dimethicones, one or more cyclomethicones, and combinations thereof. Exemplary silicones also include polydimethyl siloxanes end-capped with dimethylsilanol groups (e.g., dimethiconol according to the CTFA name). In one embodiment, the composition of the present invention includes a blend of cyclomethicone, dimethiconol, and dimethicone.

The formulation of the present invention also can include one or more fatty esters. Suitable fatty esters can include, for example, fatty esters of one or more polyhydric alcohol esters and combinations of one or more of such fatty esters. An exemplary polyhydric alcohol fatty ester is glyceryl monostearate.

The formulation of the present invention also can include one or more components in addition to those described above, such as, e.g., ingredients that are commonly used in formulating hair conditioners. Such additional components can include, for example, one or more thickeners, one or more preservatives, one or more fragrances, or a combination thereof, or any other ingredients that can be incorporated into the formulation of the present invention to adjust or enhance formulation characteristics such as, e.g., texture, thickness, appearance, fragrance, and the like.

The present invention further provides a method of conditioning keratinous fibers, which preferably includes applying to the keratinous fibers a conditioning-effective amount of the formulation of the present invention. The keratinous fibers can include mammalian hair, e.g., human hair. The formulation can be applied in any suitable manner, e.g., by working the composition through hair, e.g., with the hands and fingers or with a suitable implement such as, e.g., a comb or brush, to ensure good coverage. The composition can be left on the hair for any suitable length of time, e.g., to allow the composition to be incorporated into the hair shaft. The formulation of the present invention can be applied to dry hair, wet hair or damp hair, e.g., following shampooing, and can be left on the hair for, e.g., from about 10 seconds to about 5 minutes or longer if desired. Following application, the formulation can be removed, e.g., by rinsing with water sufficiently to remove at least a portion of the formulation, e.g., to remove any excess conditioner formulation, which is not incorporated into the hair shaft.

The following example further illustrates the invention but, of course, should not be construed as in any way limiting its scope.

### EXAMPLE 1

This example illustrates a process for preparing an exemplary composition of the present invention.

The list of ingredients and the amount of each ingredient (in wt % relative to the total weight of the composition) of Hair Conditioning Compositions 1A, 1B, 1C are and heated summarized in Table 1. The preparation of Hair Conditioning Compositions 1A, 1B, and 1C consists of a three-part process. Deionized water is placed in an appropriately sized vessel and heated, with stirring, to 80° C. Disodium EDTA is then added. When the temperature reaches 60° C. cetyl alcohol, stearyl alcohol, glyceryl stearate, stearalkonium chloride, cetrimonium chloride, and the bis-alkoxy-terminated glycol functionalized aminosilicone are added. Next, the non-functionalized aminosilicone is added and is mixed at high speed at a temperature of 80° C. for 25 minutes. After mixing for 25 minutes, the mixture is cooled to 50° C. and the pre-blend of silicones is added. This is then mixed at high speed at 50° C. for 20 minutes. Next, another portion of water is added and is followed by the addition of DMDM hydantoin. Finally, when the mixture has cooled to 42° C. to 44° C., the fragrance is added to the mixture.

TABLE 1

| Hair Conditioning | | | |
|---|---|---|---|
| Composition | 1A (wt. %) | 1B (wt. %) | 1C (wt. %) |
| Water | 86.90 | 87.40 | 86.90 |
| Disodium EDTA | 0.05 | 0.05 | 0.05 |
| Cetyl Alcohol | 3.50 | 3.50 | 3.50 |
| Stearyl Alcohol | 2.00 | 1.50 | 2.00 |
| Glyceryl Stearate | 0.60 | 0.60 | 0.60 |
| Stearalkonium Chloride | 0.60 | 0.60 | 0.60 |
| Cetrimonium Chloride (30%) | 1.30 | 1.30 | 1.30 |
| Dow Corning 8500 | 1.00 | 1.00 | 1.00 |
| Abil Soft AF-100 | 0.50 | 0.00 | 0.00 |
| Ciba Tinocare SA-1 | 0.00 | 0.50 | 0.50 |
| Dimethicone | 1.00 | 1.00 | 1.00 |
| Cyclomethicone | 2.00 | 2.00 | 0.00 |
| Cyclomethicone & | 0.00 | 0.00 | 2.00 |
| Dimethiconol blend | | | |
| DMDM Hydantoin | 0.20 | 0.20 | 0.20 |
| Fragrance | 0.35 | 0.35 | 0.35 |

The formulations impart a silky wet feel to hair when applied as a rinse off conditioner and exhibit good emulsion stability under freeze-thaw conditions and/or under conditions of storage at elevated temperature (as further described in Examples 2 and 3 herein).

### EXAMPLE 2

This example illustrates the stability of exemplary formulations of the present invention. Composition 2A was prepared as described in Example 1. When submitted to a succession of three freeze-thaw cycles, composition 2A remained integrated. One freeze-thaw cycle consists of freezing the sample overnight in a freezer set at −20° C. and letting the sample warm up to room temperature. After three freeze-thaw cycles, compositions 1A, 1B, 1C, and 2A show no visible phase separation (whereas comparative compositions lacking an alkyl quaternary ammonium salt separate into two visible phases).

TABLE 2

| Hair Conditioning Composition | 2A (wt. %) |
|---|---|
| Water | 86.90 |
| Disodium EDTA | 0.05 |
| Cetyl Alcohol | 3.50 |
| Stearyl Alcohol | 2.00 |
| Glyceryl Stearate | 0.60 |
| Stearalkonium Chloride | 0.60 |
| Cetrimonium Chloride | 1.30 |
| Dow Corning 8500 | 1.50 |
| Silsoft A-843 | 0.50 |
| Dimethicone | 1.00 |
| Cyclomethicone | 1.50 |
| DMDM Hydantoin | 0.20 |
| Fragrance | 0.35 |

### EXAMPLE 3

This example illustrates the stability of exemplary formulations of the present n at elevated storage temperatures. Compositions 1A, 1B, 1C, 2A, 3A and 3B (as described in Example 4) were stored at 45° C. for 2 weeks and showed no sign of separation.

### EXAMPLE 4

This example demonstrates the improved conditioning performance of an ry formulation of the present invention. Compositions 3A and 3B were prepared as d in Example 1 and are described in Table 3.

TABLE 3

| Hair Conditioning | | |
|---|---|---|
| Composition | 3A (wt. %) | 3B (wt. %) |
| Water | 88.90 | 86.90 |
| Disodium EDTA | 0.05 | 0.05 |
| Cetyl Alcohol | 3.50 | 3.50 |
| Stearyl Alcohol | 2.50 | 2.00 |
| Glyceryl Stearate | 0.60 | 0.60 |
| Stearalkonium Chloride | 0.60 | 0.60 |
| Cetrimonium Chloride (30%) | 1.30 | 1.30 |
| Dow Corning 8500 | 1.50 | 1.00 |
| Silsoft A-843 | 0.50 | |
| Ciba Tinocare SA-1 | | 0.50 |
| Dimethicone | | 1.00 |
| Cyclomethicone | | |
| Cyclomethicone & Dimethiconol blend | | 2.00 |
| DMDM Hydantoin | 0.20 | 0.20 |
| Fragrance | 0.35 | 0.35 |

Composition 3B has been tested blind in a Home Use consumer test. The results found that composition 3B provided excellent results in product feel, product application, and the product's conditioning characteristics such as wet combing, dry combing, softness and shine.

Comparative compositions were prepared, which lacked the bis-alkoxy-terminated glycol-functionalized aminosilicone, non-flinctionalized aminosilicone, and the cyclomethicone/dimethicone blend (comparative composition A) and which lacked the cyclomethicone/dimethicone blend but otherwise contained the bis-alkoxy-terminated glycol-functionalized aminosilicone and non-functionalized aminosilicone (comparative composition B). Composition B exhibited an improvement in conditioning performance over comparative composition A, and composition 3A exhibited an improvement in conditioning performance over comparative composition B, on bleached blond 5 gm tresses (purchased from International Hair Importer). The tresses are first shampooed twice with VO5 Normal shampoo and rinsed under tap warm (35-40° C.) water then conditioned with 1.5 ml of conditioner. Progressive improvement in the slickness feel during the application as well as the feel and ease of wet and dry combing is observed from comparative composition A, to comparative composition B, to composition 3A. The results show an increased conditioner performance upon the addition of the amodimethicone to the formulation. Formulation 3A outperformed comparative composition B, a formulation that included non-functionalized silicones but did not contain any amodimethicones. Both formulations 3A and comparative composition B outperformed comparative composition A, which contained neither an amodimethicone nor a non-funcitonalized silicone.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g.,“such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A conditioning composition comprising one or more bis-alkoxy-terminated glycol-functionalized aminosilicones, one or more non-functionalized aminosilicones, one or more fatty alcohols, one or more alkyl quaternary ammonium salts, and water.

2. The composition of claim 1, wherein the one or more bis-alkoxy-terminated glycol-functionalized aminosilicones comprises a bis-C₁₃-C₁₅-alkoxy-terminated glycol-functionalized aminosilicone.

3. The composition of claim 1, wherein the one or more bis-alkoxy-terminated glycol-functionalized aminosilicones comprises a bis-C₁₃-C₁₅-alkoxy-terminated propylene glycol-functionalized aminosilicone.

4. The composition of claim 1, wherein the one or more bis-alkoxy-terminated glycol-functionalized aminosilicones comprises a copolymer of the formula: 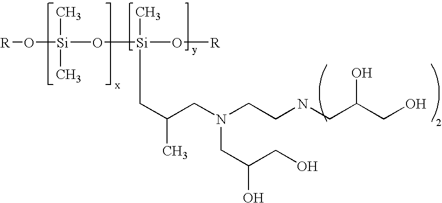
wherein each R is independently a C₁₃-C₁₅ alkyl, x is from about 25 to about 1000, and y is from 1 to about 200, and x and y are chosen such that the nitrogen content is greater than about 0.5%.

5. The composition of claim 1, wherein the one or more non-functionalized aminosilicones comprises:
a compound of the formula: 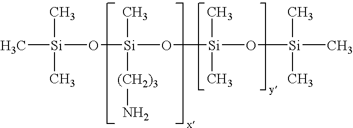
wherein the x′:y′ ratio is about 1:50, and the molecular weight of the compound is at least about 16,000;
a compound of the formula: 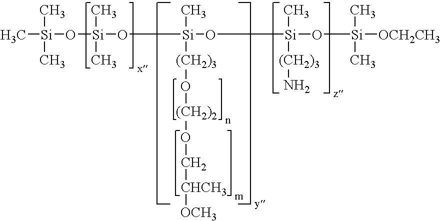
wherein x″ is from about 20 to about 100, y″ from about 2 to about 10, z″ is from 1 to about 5, m is from about 3 to about 5, and n is from about 5 to about 9;
a polyoxyalkylenated aminosilicone polymer of the (AB)ₙ type, wherein A is a polysiloxane block and B is a polyoxyalkylenated block, comprising repeating units of the formulae: [SiMe₂-O—(SiMe₂O)_{x′″}SiMe₂-R′—N(H)—R″—O(C₂H₄O)ₐ—(C₃H₆O)_{b}—R″—N(H)—R′—],
a is from about 1to about 200,
b is from 0 to about 200,
R′, which may be identical or different, is a divalent organic group which is linked to the adjacent silicon atom via a carbon-silicon bond and to the nitrogen atom,
R″, which may be identical or different, is a divalent organic group which is linked to the adjacent oxygen atom via a carbon-oxygen bond and to the nitrogen atom, and
x′″ is chosen such that the siloxane block represents between 50 and 95 mol % relative to the total weight of the polyoxyalkylenated aminosilicone polymer;
or a combination thereof.

6. The composition of claim 5, wherein the one or more non-functionalized aminosilicones comprises a compound of the formula:

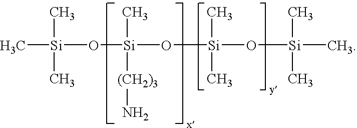

7. The composition of claim 1, wherein the one or more fatty alcohols comprises one or more C₁₂-C₂₀ alcohols.

8. The composition of claim 1, wherein the one or more fatty alcohols comprises one or more C₁₆-C₁₈ alcohols.

9. The composition of claim 1, wherein the one or more fatty alcohols comprises cetyl alcohol, stearyl alcohol or a combination thereof.

10. The composition of claim 1, wherein the one or more fatty alcohols are present in the composition in an amount of from about 5.0 wt % to about 6.0 wt %.

11. The composition of claim 1, wherein the one or more alkyl quaternary ammonium salts comprises a compound of the formula: 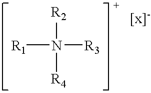
wherein R₁ and R₂ are the same or different and each is a linear or branched aliphatic substituent containing from 1 to about 30 carbon atoms, and R₃ and R₄ are the same or different and each is a linear or branched alkyl substituent containing from 1 to about 3 carbon atoms or an aralkyl substituent containing at least 7 carbon atoms, and X is an anion.

12. The composition of claim 1, comprising from about 60 wt % to about 95 wt % water.

13. The composition of claim 1, further comprising one or more dimethicones, one or more hydroxyl-terminated dimethicones, one or more cyclomethicones, or a combination thereof.

14. The composition of claim 1, further comprising one or more fatty esters.

15. The composition of claim 1, further comprising one or more thickeners, one or more preservatives, one or more fragrances, or a combination thereof.

16. A method of conditioning keratinous fibers, the method comprising contacting the keratinous fibers with the composition of claim 1 in an amount effective to condition the keratinous fibers.

17. The method of claim 16, further comprising rinsing off the keratinous fibers after contacting the keratinous fibers with the composition.

18. The method of claim 16, wherein the keratinous fibers comprise mammalian hair.

19. The method of claim 16, wherein the keratinous fibers comprise human hair.
